(19) United States
(12) Patent Application Publication
(10) Pub. No.: US 2007/0298130 A1
(43) Pub. Date: Dec. 27, 2007

(54) Convolvulus Soldanella Compositions and Methods of Use

(76) Inventor: Ba X. Hoang, Hayward, CA (US)

Correspondence Address: PERKINS COIE LLP, P.O. BOX 2168, MENLO PARK, CA, 94026, US

(21) Appl. No.: 10/592,990

(22) PCT Filed: Mar. 17, 2005
(86) PCT No.: PCT/US05/09019

§ 371 Date: Jun. 12, 2007

Related U.S. Application Data
(60) Provisional application No. 60/554,112, filed on Mar. 17, 2004.
(60) Provisional application No. 60/588,848, filed on Jul. 15, 2004.

(51) Int. Cl. A61K 35/00; A61P 17/00; A61P 35/00

(52) U.S. Cl. 424/725

(57) ABSTRACT

Compositions and methods for treating patients suffering from a disorder characterized by an increased angiogenesis, an inflammatory skin disease, and/or to effect weight loss or treat obesity are described. Included are compositions comprised of an extract of sea bindweed (Convolvulus soldanella).

### FIELD OF THE INVENTION

The present invention relates to compositions for inhibiting angiogenesis, for treatment of skin conditions including eczema and psoriasis, and for promoting weight loss and treating obesity. The compositions further find use in treating cancer or other proliferative disorders as an angiogenic inhibitor. The composition comprises an extract of Convolvulus soldanella.

### REFERENCES

Brem, et al., J Pediatr Surg, 28(3):445-450 (1993).
Ciardella, et al., Ophthalmol Clin North Am., 15(4):453-458 (2002).
Crandall et al., Microcirculation, 4:211-232 (1997).
D'Amore, Semin Thromb Hemost, 14(1):73-78 (1988).
Folkman, Important Adv Oncol, pp. 42-62, 1985.
Griscelli, et al., PNAS USA, 95(11):6367-6372 (1998).
Hausman et al., J. Anim. Sci., 82:925-934 (2004).
Hayes, Hematol Oncol Clin North Am, 8(1):51-71 (1994).
Hooper, et al., Clin Experiment Ophthalmol, 31(5):376-391 (2003).
MacDonald, www.childhoodbraintumor.org.
Meng, et al., U.S. Pat. No. 6,083,510, issued Jul. 4, 2000.
Meng et al., PR Health Sci J, 21(4):323-328 (2002).
Mousa, et al., Curr Pharm Des, 10(1):1-9 (2004).
Nguyen et al., Microvasc Res, 41(1):31-40 (1994).
Pluda, Semin Oncol, 24(2):203-218 (1997).
Rupnick, et al., PNAS USA, 99(16):10730-10735 (2002).
Scappaticci, Expert Opin Investig Drugs, 12(6):923-932 (2003).
Sierra-Honigmann, et al., Science, 281:1683-1686 (1998).
Yoshida, et al., Histol Histopathol, 14(4):1287-94 (1999).

### BACKGROUND OF THE INVENTION

Convolvulus soldanella, also called Calystegia soldanella, sea bindweed, or seashore false bindweed is a perennial dicotyledonous vine of the morning-glory family. Sea bindweed typically grows on sandy sea-shores. Sea bindweed has fleshy, shiny, leaves that are kidney-shaped and are without epidermal hairs. The blooms are trumpet shaped, and deep pink about 5 cm in diameter.

Sea bindweed has been used historically as a diuretic, laxative, purgative, and to treat scurvy. Other members of the Convolvulus family have been used as a purgative (www.ars-grin.gov). Additionally, high molecular extracts of field bindweed (Convolvulus arvensis) have been suggested for use as an anti-angiogenic.

### SUMMARY OF THE INVENTION

In one aspect, the invention includes a method for treating a person suffering from a cellular proliferation disorder characterized by increased angiogenesis. The method includes administering to the person a composition comprising an extract of sea bindweed. The administration can be performed on a schedule selected from daily administration, twice daily administration, and thrice daily administration. In an exemplary embodiment, the compound is administered at a daily dose of between about 500 mg to about 2000 mg. In another embodiment, the compound is administered at least once daily for a period of between about one week to about 4 months.

In an embodiment, the composition is administered by oral administration and/or parenteral administration.

The cellular proliferation disorder may be selected from the group consisting of colorectal cancer, breast cancer, lung cancer, ovarian carcinoma, osteocarcinoma, cancers of the head, and neck cancer.

In another aspect, the invention includes a composition for treating a cellular proliferation disorder characterized by increased angiogenesis. The composition comprises an extract of sea bindweed, and derivatives thereof, and

a physiologically acceptable carrier. The cellular proliferation disorder may be selected from the group consisting of colorectal cancer, breast cancer, lung cancer, ovarian carcinoma, osteocarcinoma, cancers of the head and neck cancer. The cellular proliferation disorder may further be age-related macular degeneration.

In another aspect, the present invention includes a method for treating a person suffering from an inflammatory skin condition. The method includes administering to the person a composition comprising an extract of sea bindweed. The administration can be performed on a schedule selected from daily administration, twice daily administration, and thrice daily administration. In an exemplary embodiment, the compound is administered at a daily dose of between about 500 mg to about 2000 mg. In another embodiment, the compound is administered at least once daily for a period of between about one week to about 4 months.

In an embodiment, the composition is administered by oral administration and/or parenteral administration.

The inflammatory skin condition may be eczema and/or psoriasis.

In yet another aspect, the invention includes a composition for treating an inflammatory skin condition. The composition includes an extract of sea bindweed, and derivatives thereof, and a physiologically acceptable carrier. The inflammatory skin condition may be eczema and/or psoriasis.

In another aspect, the present invention includes a method for effecting weight loss or treating obesity. The method includes administering to the person a composition comprising an extract of sea bindweed. The administration can be performed on a schedule selected from daily administration, twice daily administration, and thrice daily administration. In an exemplary embodiment, the compound is administered at a daily dose of between about 500 mg to about 2000 mg. In another embodiment, the compound is administered at least once daily for a period of between about one week to about 4 months.

### DETAILED DESCRIPTION OF THE INVENTION

### I. DEFINITIONS

“Angiogenesis” as used herein refers to the formation and differentiation of blood vessels.

“Anti-angiogenesis” as used herein refers to inhibition or reduction of angiogenesis.

“Anti-angiogenesis therapy” refers to the treatment of cancer and tumors by inhibiting angiogenesis stimulated by the cancer or tumor. The term further refers to the amelioration of the symptoms of cancer or reduction of tumor size.

“Aqueous” as used herein refers to a solution including water. Examples of aqueous extraction solvents include distilled water, and mixtures of water with ethanol, phenol, and other alcohols.

“Cancer” as used herein refers to a malignant growth characterized by uncontrolled cell proliferation. This includes both malignant and benign solid-tumor forming cancers as well as leukemia.

An “extract” as used herein refers to the product, as an essence or concentrate, prepared any suitable method for extraction, including, but not limited to cold or hot aqueous extraction. The extract further may be a solution (aqueous or other suitable solvent) of essential constituents of a complex material (the Convolvulus soldanella) prepared the extraction method.

A “skin condition” or “skin condition characterized by inflammation” as used herein refers to a skin condition characterized by redness, localized heat, swelling, pain, and itching.

“Solid-tumor” as used herein refers to an abnormal mass of tissue that usually does not contain cysts or liquid areas. Solid-tumors include benign and malignant tumors. Examples include sarcomas, carcinomas, and lymphomas.

The term “treatment” refers to inhibiting or arresting the development of a disease or condition in a patient, particularly a human, causing regression of the disease or condition, or relieving the symptoms associated with the disease or condition.

“Obesity” refers to a patient with a body mass index (BMI) greater than 30. “Treating obesity” includes prophylaxis as well as alleviation of established obesity. In addition to the treatment of obesity, the term contemplates treatment of conditions associated with obesity and conditions exacerbated by the state of being obese.

“Body Mass Index” or “BMI” refers to a measurement tool used to determine excess body weight. BMI is calculated with the formula [weight (lb.)/(height (in))²* 704.5.

### II. COMPOSITIONS AND METHODS FOR ANTI-ANGIOGENESIS THERAPY, TREATMENT OF SKIN CONDITIONS, TREATMENT OF OBESITY, AND EFFECTING WEIGHT LOSS

In one aspect, the invention provides methods and compositions for treating cancer and/or tumors characterized by excessive angiogenesis.

In another aspect, the invention provides simple, inexpensive, and safe methods and compositions for treating patients suffering from a skin condition such as eczema or psoriasis.

In another aspect, the invention provides methods and compositions for promoting weight loss, preventing weight gain, and of treating conditions associated with, exacerbated by, or directly caused by the state of being overweight or obese.

A. Compositions

The composition is one that includes an extract of sea bindweed, Convolvulus soldanella (also called Calystegia soldanella). As illustrated below, this compound has been found to be effective as an anti-angiogenic agent. As further illustrated below, this compound has been found to effectively reverse or reduce the symptoms of inflammatory skin conditions such as eczema and psoriasis. As also illustrated below, this compound has been found to effectively treat obesity or effect weight loss.

Sea bindweed is a perennial dicotyledonous vine of the morning-glory family that grows on sandy sea-shores. It has been used as a diuretic, laxative, purgative, and to treat scurvy (www.ars-grin.gov).

1. Anti-Angiogenic

Angiogenesis refers to the formation and the growth of new blood vessels. Angiogenesis occurs in the healthy body for healing wounds and for restoring blood flow to tissues after injury. The healthy body controls angiogenesis through angiogenesis-stimulating growth factors and angiogenesis inhibitors. Many disease states, such as cancer, diabetic blindness, age-related macular degeneration, rheumatoid arthritis, and psoriasis, are characterized by increased or excessive angiogenesis. Increased angiogenesis refers to angiogenesis greater than that in a normal body, especially angiogenesis in an adult not related to normal angiogenesis (menstruation, or near wounds or injuries). Excessive angiogenesis refers to angiogenesis exceeding that present in a normal body, especially angiogenesis in an adult not related to normal angiogenesis. Normal angiogenesis is infrequent in adults and generally refers to forming new blood vessels in the lining of the uterus during the menstrual cycle in women and for the repair or regeneration of tissue during wound healing. Abnormal angiogenesis can provide new blood vessels that feed diseased tissues and/or destroy normal tissues, and in the case of cancer, the new vessels can allow tumor cells to escape into the circulation and lodge in other organs (tumor metastases) (Angiogenesis Foundation, www.angio.org).

Angiogenesis inhibitors are agent or drugs that block the development of new blood vessels. Without being limited as to theory, it is thought that most anti-angiogenic agents work by preventing the growth of the endothelial cells that form the inner lining of the blood vessels, which prevents formation of new blood vessels (Griscelle et al., 1998). Other anti-angiogenic agents work by signaling cascade or by blocking the ability of endothelial cells to break down the extracellular matrix (National Cancer Institute). Anti-angiogenic agents may further target vascular endothelial growth factor (VEGF), receptors for VEGF, integrins, matrix metalloproteinases, and other blood vessel targets (Scappaticci, 2003).

As a tumor develops and grows, new blood vessels must be formed to support the tumor's growth with a supply of oxygen and nutrients to the tumor. It has been suggested to use angiogenesis inhibitors in the treatment of solid-tumors and macular degeneration (MacDonald, www.childhoodbraintumor.org). Without new blood vessels, a tumor can't grow larger than about 2 millimeters (Folkman, 1985). Additionally, large tumors may shrink if the blood supply is cut off or reduced (www.cancer.org).

Intraocular neovascularization has further been shown to be a factor in age related macular degeneration (AMD) (Yoshida, et al., 1999). AMD is caused by the deterioration of the macula, which is responsible for focusing central vision in the eye. The macula controls the ability to read, drive a car, recognize faces or colors, and see objects in fine detail. Age-related macular degeneration is the leading cause of blindness in the United States and many European countries with the neovascular form of the disease being responsible for most (90%) of the cases of severe loss of vision (www.macular-degeneration.org).

In neovascular macular degeneration, new blood vessels are found to grow from the choroid, called choroidal neovascularization. These abnormal blood vessels can grow beneath the retina and leak blood, fats, and serum and induce macular scarring and loss of vision. Anti-angiogenic agents have been found to be effective in the treatment of neovascular AMD (Ciardella et al., 2002).

It has been suggested that the high molecular extracts of Convolvulus arvensis (field bindweed) can be used to inhibit the growth of tumor cells and blood vessels through anti-angiogenesis (Meng et al., U.S. Pat. No. 6,083,510).

As described in Example 8, a study was conducted in support of using the composition of the invention for inhibiting angiogenesis. In this assay, compositions comprised of sea bindweed extract were compared in a chick chorioallantoic membrane (CAM) assay with C-statin, an extract of Convolvulus arvensis (Meng, et al., 2000). The test composition used in Example 8 was comprised of a 10× extract of Convolvulus soldanella prepared by water extraction of the leaves and stems followed by condensation under low pressure and dehydration to form the sea bindweed extract. Observation of the CAM assay revealed that C-statin inhibited angiogenesis by 20%, while the sea bindweed extract inhibited angiogenesis by 100%. As seen from these results, the Convolvulus soldanella extract was four times more effective than the Convolvulus arvensis extract in inhibiting angiogenesis.

Thus, the compositions of the present invention function as an angiogenesis inhibitor or anti-angiogenic compound. Compositions of the present invention may find use in treating or reducing the growth of carcinomas, sarcomas, leukemia, myelomas, and lymphomas. Exemplary cancers that can be treated with the compositions of the present invention include colorectal cancer, breast cancer, lung cancer, ovarian carcinoma, prostate cancer, pancreatic cancer, bone cancers such as osteocarcinoma, brain tumors, bladder cancer, endometrial cancer, kidney tumors, liver tumors, and cancers of the head and neck, among others. Compositions of the present invention may further prevent metastasis of these cancers and tumors. Compositions of the present invention may find use in treating proliferative disorders characterized by angiogenesis. Compositions of the present invention may further find use in treating macular degeneration.

It will be appreciated that compositions of the present invention may be used alone or in combination with other anti-angiogenic agents. The compositions of the present invention may further be used in combination with other treatments such as radiation therapy, chemotherapy, endocrine therapy, or other suitable treatments for proliferative disorders.

2. Skin Conditions

In studies conducted in support of the invention, compositions comprised of sea bindweed extract were prepared and administered to test subjects, as set forth in Examples 1-7. The test composition used in Examples 1-7 was comprised of a 30× extract of Convolvulus soldanella prepared by water extraction of the leaves and stems followed by condensation under low pressure and dehydration to form the powder used. In Example 1, 9 patients suffering from eczema (7 adults and 2 children) and 6 patients suffering from psoriasis was treated with 500 to 2000 mg (oral) of the bindweed extract two or three times daily for three months. At the end of the treatment period, all of the eczema patients and five of the psoriasis patients reported an improvement in symptoms. Most of the patients reported an improvement after three days (eczema patients) or one to two weeks (psoriasis patients) of treatment.

In another study, detailed in Example 2, a patient suffering severe eczema over 70% of the body was treated with 2000 mg of the bindweed extract composition three times daily for a period of 6-7 weeks. After treatment, the patient showed no visual evidence of the eczema.

A patient suffering from eczema, as detailed in Example 3, was treated with 2000 mg of the bindweed extract composition twice daily for two months. After treatment, the patient showed almost no visual evidence of the eczema.

As described in Example 4, a patient suffering from eczema with open sores on the scalp was treated with 2000 mg of the bindweed extract composition three times daily. After three weeks of treatment, the patient's sores had healed.

In Example 5, a patient suffering eczema on the face and lower legs was treated with 500 mg of the bindweed extract composition three times daily. The patient's symptoms improved after one week of treatment and after two months of treatment showed no visual sign of the eczema.

As detailed in Example 6, a patient suffering from psoriasis over 70% of the body was treated with 2000 mg of bindweed extract, three times daily. The patient's symptoms were improved after five days of treatment and after three months of treatment showed almost no visual sign of the psoriasis.

A patient suffering from severe psoriasis with 80% skin cover, as detailed in Example 7, was treated with 2000 mg of the bindweed extract three times daily. After three months of treatment, the patient's symptoms were in remission and the patient showed no visual signs of the psoriasis.

It will be appreciated that the treatment compound may be administered in combination with one or more known methods for treating inflammatory skin conditions, as detailed below. It will further be appreciated that the compound may contain buffers, solvents, etc. as known in pharmaceutical compositions.

a. Eczema

Eczema includes certain kinds of dermatitis that involves an inflammation of the skin. Symptoms include dryness, flakiness, heat, blisters, and itching. In mild forms the skin is dry, hot and itchy, while in more severe forms the skin can become broken, raw and bleeding. Examples of eczema include atopic eczema, allergic contact dermatitis, irritant contact dermatitis, seborrheic dermatitis, varicose eczema, and discoid eczema, which are further described below. The causes of eczema are many and varied, and depend on the particular type of eczema. Causes include heredity, allergens, irritants such as chemicals and detergents, allergens such as nickel, and yeast growths. In later years, varicose eczema may be caused by blood circulatory problems and/or deficiency in the legs. Other factors such as environmental factors and stress may also cause eczema (www.medinfo.co.uk).

Currently, there are a number of ways to manage eczema, including emollients, topical and oral steroids, topical immunomodulators such as tacrolimus and pimecrolimus, UV light, and anti-histamines, creams, ointments, and shower and bath oils for moisturizing the skin, evening primrose oil supplements, phototherapy, immune system suppressants including cyclosporin, and antibiotics for infections resulting from eczema (www.medinfo.co.uk).

The most common types of eczema include:

(i). Atopic Eczema

Atopic eczema is the most common form of eczema affecting about 10% of infants and about 3% of adults in the United States. One of the most common symptoms of atopic eczema is its itchiness (or pruritis), which can be extremely severe. Other symptoms include overall dryness of the skin, redness and inflammation. Scratching the inflammation can cause the skin to split, leaving it prone to infection. If infected, the skin may crack and weep (known as ‘wet’ eczema). These rashes occurs mainly on the face and scalp in infancy and on the hands and feet in teens and adults, however, patches can appear anywhere. Although these are the most common sites, any area such as the bends of the elbows, backs of the knees, ankles, wrists, face, neck, and upper chest may also be affected (www.holistichealthtools.com).

Atopic eczema is thought to be a hereditary condition, being genetically linked. People with atopic eczema may further be sensitive to allergens in the environment which are harmless to others. In atopy, there is an excessive reaction by the immune system producing the skin inflammation. Asthma and hayfever may further be associated with atopy (www.holistichealthtools.com).

(ii). Allergic Contact Dermatitis

Allergic contact dermatitis develops when the body's immune system reacts to a substance in contact with the skin. This allergic reaction often develops over a period of time through repeated contact with the substance. For example, an allergic reaction may occur to nickel, which is often found in earrings, belt buckles and jeans buttons. Reactions can also occur after contact with other substances such as perfumes and rubber (www.holistichealthtools.com).

(iii). Irritant Contact Dermatitis

Irritant contact dermatitis is a form of eczema caused by frequent contact with everyday substances, such as detergents and chemicals, which are irritating to the skin. It most commonly occurs on the hands of adults and can be prevented by avoiding the irritants and keeping the skin moisturized (www.holistichealthtools.com).

(iv). Infant Seborrhoeic Eczema

Infant seborrhoeic eczema is a common condition affecting infants under one year of age, although the exact cause is unknown. Also referred to as cradle cap, it usually starts on the scalp or the nappy area and can quickly spread to other parts of the body. Although this type of eczema looks unpleasant, it does not usually include the symptoms of soreness or itch. Infant seborrhoeic eczema normally clears up after a few months without recurrence. The use of moisturizing creams and bath oils has been found to speed the recovery (www.holistichealthtools.com).

(v). Adult Seborrhoeic Eczema

Adult seborrhoeic eczema characteristically affects adults between the ages of 20 and 40. It is usually seen on the scalp as mild dandruff, but can spread to the face, ears and chest. The skin becomes red, inflamed and starts to flake. The condition is believed to be caused by a yeast growth (www.holistichealthtools.com).

(vi). Varicose Eczema

Varicose eczema affects the lower legs of adults in their middle to late years, and is generally caused by poor circulation. Commonly the skin around the ankles is affected, becoming speckled, itchy and inflamed. Untreated skin can break down, resulting in an ulcer (www.holistichealthtools.com).

(vii). Discoid Eczema

Discoid eczema is usually found in adults and appears suddenly as a few coin shaped areas of red skin, normally on the trunk or lower legs. The areas become itchy and can weep fluid (www.holistichealthtools.com).

b. Psoriasis

Psoriasis is a chronic immune-mediated, genetic disease manifesting in the skin and/or the joints affecting more than 7 million in the United States (www.skincarephysicians.com). A family association exists in one out of three cases. Psoriasis is a non-contagious and life-long skin disease that has different forms. Symptoms often develop between the ages of 15 and 35, although they can develop at any age (health.yahoo.com). Symptoms include patches of raised red skin covered by a flaky white buildup. In certain kinds of psoriasis, symptoms include a raised, red area (pustular psoriasis) or burned (erythrodermic) appearance. Other symptoms include intense itching and burning. Some patients may have a spot or two, while others may have extensive coverage on their body. In general, symptoms are frequently found on the knees, elbows, scalp, hands, feet, or lower back. Physically, if less then 2 percent of the body is involved, the case is considered mild. Between 3 and 10 percent is considered moderate, and more than 10 percent is classified as severe (www.psoriasis.org).

There are generally five different types of psoriasis. The most common form of psoriasis (about 80%) is called “plaque psoriasis,” which is characterized by well-defined patches of red, raised skin from parakeratosis (www.psoriasis.org). Parakeratosis refers to the increased production and movement of skin cells. In normal skin, the outer layer, made up mostly of cells called keratinocytes, is replaced every 27 to 28 days with newly formed keratinocytes. With parakeratosis, this process is sped up to about 3 or 4 days. These cells are moved to the surface faster than they can be incorporated in the skin layer, thus the keratinocytes accumulate and form scaling or lesions. Other symptoms include dilated small blood vessels and inflammatory cells (www.skincarephysicians.com). Plaque psoriasis can appear on any skin surface, although the knees, elbows, scalp, trunk and nails are the most common locations. The other types of psoriasis are guttate, which presents as small, red, individual drops on the skin; inverse smooth, which presents as dry areas of skin, often in folds or creases, that are red and inflamed but do not have scaling; erythrodermic periodic, which presents as widespread, fiery redness of the skin; and pustular, which involves either generalized, widespread areas of reddened skin, or localized areas, particularly the hands and feet (palmo-plantar pustular psoriasis) (www.psoriasis.org).

Current methods of treatment include moisturizing creams and lotions, synthetic vitamin D, coal tar, anthralin, topical and oral steroids, phototherapy, UV light therapy, retinoids, PUVA (psoralen and UVA light therapy), methotrexate, immunosuppressants such as cyclosporine, and cortisone compounds (www.aad.org).

3. Weight Loss and Obesity

Obesity involves an excessive accumulation of body fat and is widely considered to be a major public health problem, associated with substantially increased morbidity and mortality, as well as psychological problems, reduced economic achievement, and discrimination. Obesity is the second leading cause of preventable death in the U.S. and currently more than half of the adult population is overweight (having a body mass index (BMI) of 25 to 29.9) and almost one third of the population is considered obese (BMI greater than or equal to 30). While obesity alone is a serious health concern, it also is known to contribute, cause or exacerbate other health problems. These problems include high blood pressure, diabetes (type 2), heart disease, stroke, gallbladder disease, cancer of the breast, prostate and colon, obstructive sleep apnea, gout, hyperlipidemia, osteoarthritis, reduced fertility, impaired psychosocial function, reduced physical agility and increased risk of accidents, and impaired obstetrical performance.

A recent study (Rupnick et al.) showed that obese mice treated with anti-angiogenic agents resulted in dose-dependent, reversible weight reduction and adipose tissue loss. Adipose tissue, unlike most adult tissues, can grow and regress throughout adulthood. This tissue is highly vascularized and has angiogenic properties (Crandall et al. and Sierra-Honigmann et al.). The study further showed adaptations in food intake, metabolic rate, and preferred energy substrate in the treated mice. It is thought that the anti-angiogenesis agents destroy preexisting blood vessels near the adipose tissue leading to destruction of the cells. As described above in relation to angiogenesis and described in Example 8, a bindweed extract was effective to inhibit angiogenesis in a CAM assay by 100%.

As seen in Example 9, five patients treated with 2000 mg of bindweed extract twice per day had a minimum weight loss of 5.6 pounds after ninety days. The patients lost a combined total of 52.8 lb. after the 90 days. For comparison, a control study was performed where five patients were treated with 2000 mg of vegetable powder twice per day. The control patients had a minimum weight loss of 1.2 pounds with a combined total of 4.6 lb. after the 90 days. The patients treated with the bindweed extract further showed a marked decrease in blood pressure (27 points systolic, 18 points diastolic), whereas the control patients showed some decrease (10 points systolic, 4 points diastolic). Additionally, patients treated with the bindweed extract had 30 points lower blood sugar compared to 6 points lower for the control.

Thus, the compositions of the present invention function to promoting weight loss. Compositions of the present invention may find use in promoting weight loss, preventing weight gain, and treating conditions associated with, exacerbated by, or directly caused by the state of being overweight or obese.

It will be appreciated that compositions of the present invention may further be used in combination with other treatments such as dietary therapy, drug therapy (including orlistat and phenylpropanolamine), appetite suppressants (including phentermine, enfluramine, sibutramine, exercise, behavior therapy, surgery, dietary supplements, liposuction, or other suitable treatments for weight loss. It will further be appreciated that compositions of the present invention may further be used in combination with other anti-angiogenic agents.

B. Administration

In one aspect, the invention provides a composition for inhibiting angiogenesis. As illustrated in Example 8, discussed above, the composition is effective in inhibiting angiogenesis. Accordingly, the invention contemplates a method of inhibiting angiogenesis and thus arresting tumor growth, reducing tumor size, and preventing metastasis. Accordingly, a composition containing sea bindweed extract is formulated into a preparation suitable for inhibiting angiogenesis.

In another aspect, symptoms associated with skin conditions such as eczema and psoriasis are treated or alleviated by the method described herein. In accord with this aspect of the invention, a composition containing sea bindweed extract is formulated into a preparation suitable for administration to a patient suffering from a skin condition such as eczema or psoriasis.

As illustrated by the studies set forth in Examples 1-7, discussed above, the compositions effectively arrest the symptoms associated with skin conditions such as eczema and psoriasis and cause a reduction in these symptoms. Accordingly, the invention contemplates a method of treating skin conditions such as eczema and psoriasis by administering a composition comprising an extract of sea bindweed in an amount effective to arrest the symptoms associated with these skin conditions. The bindweed extract may be administered alone or in combination with conventional therapies.

In yet another aspect, the invention provides a composition and method for promoting weight loss, preventing weight gain, and treating conditions associated with, exacerbated by, or directly caused by the state of being overweight or obese. In accord with this aspect, a composition containing sea bindweed extract is formulated into a preparation suitable for administration to an overweight or obese patient.

As illustrated in Example 9, discussed above, the compositions are effective to promote weight loss. Accordingly, the invention contemplates a method of effecting weight loss, which can be used to treat overweight or obese patients.

These compositions can be formulated for any desired mode of administration, discussed in more detail below. Solid, liquid, and semi-solid preparations are contemplated and are readily prepared by those of skill in the art.

As described above, the composition for treatment includes an extract from sea bindweed, Convolvulus soldanella. The extract may be prepared from the plant by any known methods. Preferably, 500 to 6000 mg of the extract is administered one to three times daily. In another embodiment, the crude, dried plant may be administered directly.

A preferred method of preparing the extract is aqueous extraction, more preferably water extraction. In one embodiment of this method, an aqueous solvent, preferably distilled water is added to the leaves and/or stems of the Convolvulus soldanella plant. The leaves and stems may be shredded, reduced to fine particles, or homogenized, by known methods including using a food processor or blender. The leaves and/or stems may be fresh or dried. The plant parts are agitated, pressed, and/or crushed in the aqueous solution. The plant parts are then filtered from the resulting extract by known means and the supernatant is retained. The extract may also be prepared by a similar process using a heated aqueous solvent. Preferably, a boiling or near-boiling aqueous solution is used. The extract may further be prepared as a decoction by adding the aqueous solvent, adjusting the heat to bring the mixture to a boil or near boil, reducing the heat, and simmering for 10-15 min. In another embodiment, the extract is prepared by boiling the plant parts in the aqueous solvent for 10-30 min. In yet another embodiment, the aqueous solvent is added to the plant stems and leaves, the mixture is boiled with stirring and the insoluble matters are removed by filtration or the like to give an extract solution. The extract solution is lyophilized or otherwise concentrated if necessary and spray-dried or freeze-dried in vacuo to prepare a powder as described in U.S. Pat. No. 5,576,241. Alternatively, the extract solution is condensed under low pressure and the solution is dehydrated by any known means, typically freeze drying to prepare a solid. This process may be repeated to prepare a concentrated extract. In yet another embodiment, the fresh juice of the plant is used to prepare the extract. Additionally, the extract may be prepared by any known methods of extraction known in the art, as exemplified by the methods described in Cho et al. Planta Medica, 5:343-345, (1986), incorporated by reference herein.

Routes of delivery include, but are not limited to, various systemic routes, including oral and parenteral routes, e.g., intravenous, subcutaneous, intraperitoneal, and intramuscular, as well as inhalation and transdermal delivery. Administration via these routes is achieved by formulating the compositions into a suitable dosage form. Non-limiting examples include pills, tablets, capsules, suspensions, syrups, buccal/mucosal patches, gels, ointments, suppositories, and the like. Preparation of such dosage forms is routine to those of skill in the art and exemplary references describing preparation of extracts, decoctions, pills, and suspensions are known, such as Chinese Herbal Medicine: Materia Medica; Dan Bensky and Andrew Gamble, ed.; Eastland Press, Seattle, c1986, which is incorporated herein in its entirety. In a preferred embodiment, the composition is administered orally.

Parenteral administration includes injection or gradual infusion over time. The compounds of the invention can be injected intravenously, intraperitoneally, intramuscularly, intratumorally, intranasal or administered transdermally.

The composition may be administered directly to a subject or in a suitable pharmaceutical carrier. In one embodiment, the composition is administered with a physiologically acceptable carrier, excipient, or diluent, where the composition is dissolved or dispersed therein as an active ingredient and formulated according to conventional practice. The carrier may be any of a variety of standard physiologically acceptable carrier employed by those of ordinary skill in the art. It will be understood that the choice of suitable physiologically acceptable carrier will vary dependent upon the chosen mode of administration.

Sustained release compositions are also contemplated within the scope of the invention. These may include semipermeable polymeric matrices in the form of shaped articles such as films or microcapsules.

In one embodiment, the composition may be administered at regular intervals, e.g., daily, two times daily or three times daily. In another embodiment, the composition is administered over a period of time, e.g. 3 to 6 to 12 months or more. It will be appreciated that administration of the composition may be continued for an indefinite time period. It will be appreciated that dosages of the composition will vary dependent upon the compound used in the composition. Preferred doses for oral administration of the extract are from about 500 mg to 6000 mg on a daily basis.

Dosages will vary in accordance with such factors as the age, health, sex, size and weight of the patient, the route of administration, and the efficacy of the compound. Greater or lesser amounts of the compound may be administered as required.

It will be appreciated that other herbs or ingredients may be administered with the treatment compound. It will further be appreciated that other treatment methods may be used in combination with administration of the treatment compound.

### III. EXAMPLES

Materials and Methods

Preparation of Treatment Composition

A 30× Convolvulus soldanella extract (bindweed extract) was prepared by water extraction of the dried leaves and stems of the plant. The extract was condensed under low pressure and dehydrated.

### Example 1

Treatment with the composition was studied in 15 cases of patients aged 4 years to 68 years diagnosed with eczema or psoriasis (7 adults and 2 children with eczema, 6 adults with psoriasis). Before the trial, the patients recorded the severity of dryness and inflammation for the eczema and the severity of the scaling for the psoriasis on a five point scale (1-symptoms not present; 2-very mild symptoms; 3-mild symptoms; 4-severe symptoms; 5-very severe symptoms). The results are presented in Table 1.

TABLE 1
Patients presenting severe or very severe symptoms

| | Number of Patients | Percentage (%) |
|---|---|---|
| Symptoms of eczema | | |
| Dryness | 7 | 78.0 |
| Inflammation | 9 | 100.0 |
| Dryness and | 6 | 67.0 |
| Inflammation | | |
| Symptoms of psoriasis | | |
| Thickness of scaling | 6 | 100.0 |
| Inflammation | 4 | 67.0 |
| Thickness and | 4 | 67.0 |
| Inflammation | | |

The patients were treated with 500-2000 mg of the bindweed extract, two or three times daily, on an empty stomach, for 3 months. At the end of each week, the patients recorded the severity of dryness and inflammation for the eczema and the severity of the scaling for the psoriasis on the five point scale as above. The results are summarized in Tables 2 and 3. Most of the eczema patients recorded improvement after 3 days to one week after commencement of treatment. Most of the psoriasis patients recorded improvement in symptoms after one to two weeks of treatment.

TABLE 2
Eczema treatment results

| | | | | Percentage |
|---|---|---|---|---|
| Symptoms | Adults (7) | Children (2) | Total (9) | (%) |
| Improvement | 7 | 2 | 9 | 100 |
| No change or worse | 0 | 0 | 0 | 0 |

TABLE 3
Psoriasis treatment results

| Symptoms | Patients (6 total) | Percentage (%) |
|---|---|---|
| Improvement | 5 | 83 |
| No change or worse | 1 | 17 |

### Example 2

A 59 year-old male was diagnosed with severe eczema and presented a rash and pustules over 70% of his body. The patient was treated with oral steroid with no improvement. The patient had been experiencing symptoms of eczema for approximately 10 years.

The patient was treated with 2000 mg of bindweed extract, three times daily. After two weeks of treatment, the rash and pustules had faded and dried. After 6-7 weeks of treatment, the patient showed no visual evidence of the eczema.

### Example 3

A 28 year-old female was diagnosed with eczema and presented itching, skin dryness, and pain in several places. The patient was treated with corticosterol cream with no improvement. The patient was then treated with hydrocortisol injections to control the symptoms. The patient had been experiencing symptoms of eczema for approximately 6 years.

The patient was treated with 2000 mg of bindweed extract, twice daily. After three days of treatment, the symptoms were reduced by more than 50%. At the end of four weeks of treatment, the symptoms were reduced by more than 80%. After two months of treatment, the patient showed almost no visual evidence of the eczema.

### Example 4

A 34 year-old female was diagnosed with eczema presenting open sores on the scalp. The patient was treated with 2000 mg of bindweed extract, three times daily. After one week of treatment, the sores on the scalp started healing. After three weeks all the open sores and skin eruptions were completely healed.

### Example 5

A 4 year-old male was diagnosed with eczema and presented a rash on the face and lower legs. The patient was treated with hydrocortisone cream, which relieved the symptoms temporarily.

The patient was treated with 500 mg of bindweed extract, three times daily. After one week of treatment, the symptoms were reduced by more than 70%. After two months of treatment, the patient showed no visual evidence of the eczema.

### Example 6

A 45 year-old male was diagnosed with severe psoriasis with 70% skin cover. The patient was treated with methotrexate, which relieved the symptoms temporarily.

The patient was treated with 2000 mg of bindweed extract, three times daily. The patient's skin appearance improved and symptoms were reduced after five days of treatment. After three weeks of treatment, the symptoms were reduced by more than 80%. After three months of treatment, the patient showed healthy skin with almost no visual evidence of the psoriasis.

### Example 7

A 62 year-old female was diagnosed with severe psoriasis with 80% skin cover. The patient was treated with several conventional treatments with no improvement.

The patient was treated with 2000 mg of bindweed extract, three times daily. After ten days of treatment, the symptoms were reduced by more than 50%. After two months of treatment, the psoriasis was in complete remission and the patient showed no visual evidence of the psoriasis.

### Example 8

A 10× Convolvulus soldanella extract (bindweed extract) was prepared by water extraction of the dried leaves and stems of the plant. The extract was condensed under low pressure and dehydrated.

The chick chorioallantoic membrane (CAM) assay is a widely used assay for screening angiogenesis inhibition as the early chick embryo lacks a mature immune system (Nguyen, et al. 1994). To assess the anti-angiogenic or angiogenic activity of test substances, the compounds are prepared on gelatin sponges and then implanted onto the CAM through a window cut in the shell.

Six fertilized chicken eggs of indeterminate variety were scrubbed with 70% ethanol and placed in a sterilized incubator. The eggs were rotated daily for three days. A small hole was pierced in the egg and a syringe and needle was used to remove 2 ml of albumin from the egg. Following this procedure, a band of tape was placed around the egg and a round window approximately 2 cm in diameter was cut in the egg shell. This window was sealed with tape, and the egg was returned to the incubator. The egg was allowed to incubate for a further five days, with a gentle twisting every day to move the embryo.

On day 8, the CAM tests were initiated. The sea bindweed extract to be tested was prepared by dissolving the leaves and stems in distilled water, followed by a coarse filtration through a 1 micron filter, followed by ultra filtration using a Millipore stirred cell and a 100 kDa polysulfone membrane. The retained material was washed into a tube and freeze dried to obtain a solid. This solid was dissolved in sterile water, passed through a 0.22 micron filter, and measured onto approximately 1 mm cubical sponges using a micro pipette to give a final concentration of 100 micrograms of freeze dried solid per sponge. The sponge was laid on the CAM near the Y branch of a large blood vessel. The egg was resealed and returned to the incubator for 3 days.

On day 11, the CAM was observed to determine the degree of anti-angiogenic activity of the samples. The activity was estimated by comparing a region about 4 mm in diameter surrounding the sponge to a similar region further away from the sponge. The number of branches and size of the blood vessels were counted and estimated. C-Statin inhibited angiogenesis by 20%, while the sea bindweed extract inhibited angiogenesis by 100%.

### Example 9

Ten patients aged 38-56 were divided into two groups of 5 patients. Patients in Group 1 were treated with 2000 mg of bindweed extract administered twice a day between meals continuously for 90 days. Patients in Group 2 were treated with 2000 mg of vegetable powder administered twice daily between meals continuously for 90 days as a control.

Each of the patients was measured for weight, blood sugar, and Systolic and Diastolic Blood Pressure at the end of the 90 days. The results are summarized in Table 4 below.

TABLE 4
Weight Loss Results

| | Group I | Group II |
|---|---|---|
| Total weight loss | 52.8 lb. | 4.6 lb. |
| Weight loss | 24 lb. max/5.6 lb. min | 2.8 lb. max/1.2 lb. min |
| Systolic blood pressure | 27 points lower | 10 points lower |
| Diastolic blood pressure | 18 points lower | 4 points lower |
| Blood sugar | 30 points lower | 6 points lower |

Although the invention has been described with respect to particular embodiments, it will be apparent to those skilled in the art that various changes and modifications can be made without departing from the invention.

1. A method for treating a person suffering from a cellular proliferation disorder characterized by increased angiogenesis comprising:
administering to the person a composition comprising an extract of sea bindweed.

2. The method of claim 1, wherein said administering is performed on a schedule selected from daily administration, twice daily administration, and thrice daily administration.

3. The method of claim 1, wherein said administering is performed at least once daily for a period of between about one week to about 4 months.

4. The method of claim 1, wherein said administering is performed at a daily dose of between about 500 mg to about 2000 mg.

5. The method of claim 1, wherein said administering is selected from the group consisting of oral administration and parenteral administration.

6. The method of claim 1, wherein said cellular proliferation disorder is selected from the group consisting of colorectal cancer, breast cancer, lung cancer, ovarian carcinoma, osteocarcinoma, cancers of the head and neck cancer.

7. A composition for treating a cellular proliferation disorder characterized by increased angiogenesis, comprising:
an extract of sea bindweed, and derivatives thereof; and
a physiologically acceptable carrier.

8. The composition of claim 7, wherein the cellular proliferation disorder is selected from the group consisting of colorectal cancer, breast cancer, lung cancer, ovarian carcinoma, osteocarcinoma, cancers of the head and neck cancer.

9. The composition of claim 7, wherein the cellular proliferation disorder is age-related macular degeneration.

10. A method for treating a person suffering from an inflammatory skin condition, comprising:
administering to the person a composition comprising an extract of sea bindweed.

11. The method of claim 10, wherein said administering is performed on a schedule selected from daily administration, twice daily administration, and thrice daily administration.

12. The method of claim 10, wherein said administering is performed at least once daily for a period of between about one week to about 4 months.

13. The method of claim 10, wherein said administering is performed at a daily dose of between about 500 mg to about 2000 mg.

14. The method of claim 10, wherein said administering is selected from the group consisting of oral administration and parenteral administration.

15. A composition for treating an inflammatory skin condition, comprising:
an extract of sea bindweed, and derivatives thereof; and
a physiologically acceptable carrier.

16. The composition of claim 15, wherein the inflammatory skin condition is selected from eczema and psoriasis.

17. A method for promoting or maintaining weight loss in a patient, comprising:
administering to the patient a composition comprising an extract of sea bindweed.

18. The method of claim 17, wherein said administering is performed on a schedule selected from daily administration, twice daily administration, and thrice daily administration.

19. The method of claim 17, wherein said administering is performed at a daily dose of between about 500 mg to about 2000 mg.

20. The method of claim 1, wherein said administering is selected from the group consisting of oral administration and parenteral administration.

21. A composition for promoting or maintaining weight loss, comprising:
an extract of sea bindweed, and derivatives thereof; and
a physiologically acceptable carrier.
